(19) United States
(12) Patent Application Publication
(10) Pub. No.: US 2026/0045371 A1
(43) Pub. Date: Feb. 12, 2026

(54) MEDICATION USAGE INSIGHTS BASED ON EHR DATA

(71) Applicant: Helix, Inc., San Mateo, CA (US)

(72) Inventors: Matthew Levy, Washington, DC (US); Elizabeth Cirulli Rogers, Lakeside, CA (US); Natalie Telis, San Carlos, CA (US)

(21) Appl. No.: 18/800,055

(22) Filed: Aug. 10, 2024

(51) Int. Cl. G16H 70/40; G16H 10/60; G16H 20/10; G16H 50/70

(52) U.S. Cl. CPC G16H 70/40; G16H 10/60; G16H 20/10; G16H 50/70

(57) ABSTRACT

Apparatus and method for supporting medication research. In an embodiment, an apparatus executes an algorithm to receive a request regarding a medication, obtain Electronic Health Record (EHR) data for a population of patients prescribed the medication within a target time period, perform electronic data processing on the EHR data for the population of the patients based on cohort criteria specified in the request to identify a cohort of the patients from the population, compile the EHR data for the cohort, and provide a report regarding the medication based on the compiled EHR data.

### TECHNICAL FIELD

The following disclosure relates to the field of medications, and in particular, to usage of medications.

### BACKGROUND

Pharmaceutical companies, healthcare practitioners, and/or other entities may perform research or studies on medications or prescription drugs to ensure safety of the drugs, determine the effectiveness of the drugs, etc. One issue is how to gather information for people that have taken a medication of interest in a real-world context, once the drug has received regulatory approval and is commercially available. One way to gather information may be to access pharmacy records for patients. However, pharmacy records may be incomplete or inaccessible, as separate pharmacies and providers each store their own records. Also, each set of pharmacy records at each pharmacy may require separate consent for sharing from each patient being considered, even when patient data is anonymized. Another way to gather information may be to access insurance records of patients from an insurance provider(s). However, insurance records may be similarly difficult to access and/or parse in a meaningful way. Each insurance company has its own data privacy policies and systems, and claims data is often not standardized across insurers. Thus, the ability to track medication usage of anonymized patients on a population scale remains a challenge.

### SUMMARY

Embodiments described herein provide an automated solution for gathering information on patients that have taken a medication of interest based on Electronic Health Records (EHRs). As a general overview, an apparatus referred to as a coordination server, is configured to acquire EHR data for a population of patients prescribed the medication of interest. The coordination server is configured to identify a cohort of patients based on the EHR data. More specifically, medication prescription records and other specific criteria referred to herein as cohort criteria will be used. For example, the cohort criteria may specify demographic information, phenotype information, genetic information, etc. The coordination server is therefore configured to filter the patients prescribed the medication based on the cohort criteria, resulting in the cohort of patients. The coordination server is configured to compile the EHR data for the cohort of patients, and provide a report based on the compiled EHR data. One technical benefit is the coordination server may have consent/access to EHR data of one or more healthcare providers across one or more healthcare provider networks with potentially disparate EHR systems, and is therefore able to automatically acquire the EHR data for a large population of patients. Thus, individual pharmacy records, insurance records, consents, etc., do not need to be acquired, which improves the efficiency of drug-related investigations.

In an embodiment, an apparatus comprises a network interface configured to communicate over a communication network, and a medication insight controller, comprising a processor and memory, configured to execute an algorithm to: receive a request regarding a medication via the network interface, obtain EHR data for a population of patients prescribed the medication within a target time period, perform electronic data processing on the EHR data for the population of the patients based on cohort criteria specified in the request to identify a cohort of the patients from the population, compile the EHR data for the cohort, and provide a report regarding the medication based on the compiled EHR data via the network interface. To identify the cohort, the medication insight controller is configured to implement a usage inference process to estimate medication usage of a patient during the target time period based on prescription data included in the EHR data. The usage inference process comprises processing the prescription data to determine whether the prescription data indicates multiple prescriptions of the medication during the target time period, and excluding the patient from the cohort when the prescription data indicates less than the multiple prescriptions, processing the prescription data to estimate a time gap between consecutive prescriptions, and excluding the patient from the cohort when the time gap exceeds a gap threshold, and processing the prescription data to estimate a usage duration of the medication during the target time period, and excluding the patient from the cohort when the usage duration is less than a usage threshold.

In an embodiment, a method comprises executing an algorithm, at a medication insight controller comprising a processor and memory, to perform: receiving a request regarding a medication over a communication network, obtaining EHR data for a population of patients prescribed the medication within a target time period, performing electronic data processing on the EHR data for the population of the patients based on cohort criteria specified in the request to identify a cohort of the patients from the population, compiling the EHR data for the cohort, and providing a report regarding the medication based on the compiled EHR data over the communication network. Identifying the cohort comprises implementing a usage inference process to estimate medication usage of a patient during the target time period based on prescription data included in the EHR data. The usage inference process comprises: processing the prescription data to determine whether the prescription data indicates multiple prescriptions of the medication during the target time period, and excluding the patient from the cohort when the prescription data indicates less than the multiple prescriptions, processing the prescription data to estimate a time gap between consecutive prescriptions, and excluding the patient from the cohort when the time gap exceeds a gap threshold, and processing the prescription data to estimate a usage duration of the medication during the target time period, and excluding the patient from the cohort when the usage duration is less than a usage threshold.

Other embodiments may include computer readable media, other systems, or other methods as described below.

The above summary provides a basic understanding of some aspects of the specification. This summary is not an extensive overview of the specification. It is intended to neither identify key or critical elements of the specification nor delineate any scope particular embodiments of the specification, or any scope of the claims. Its sole purpose is to present some concepts of the specification in a simplified form as a prelude to the more detailed description that is presented later.

### DESCRIPTION OF THE DRAWINGS

Some embodiments of the present disclosure are now described, by way of example only, and with reference to the accompanying drawings. The same reference number represents the same element or the same type of element on all drawings.

FIGS. 1A-1B are block diagrams of a medication insight architecture in an illustrative embodiment.

FIG. 2 is a block diagram illustrating genetic testing in an illustrative embodiment.

FIG. 3 is a flow chart illustrating a method of genetic testing in an illustrative embodiment.

FIG. 4 is a block diagram of a coordination server in an illustrative embodiment.

FIGS. 5A-5C are flow charts illustrating a method of processing EHR data for patients in an illustrative embodiment.

FIGS. 6A-6B are diagrams illustrating a request received by a coordination server in an illustrative embodiment.

FIG. 7 is a diagram illustrating cohort criteria in an illustrative embodiment.

FIG. 8 is a diagram illustrating retrieval of EHR data in an illustrative embodiment.

FIG. 9 illustrates identification of a cohort from a population of patients in an illustrative embodiment.

FIG. 10 is a diagram illustrating use of an ML system to process EHR data in an illustrative embodiment.

FIGS. 11A-11B are diagrams illustrating a coordination server providing a report regarding a medication in an illustrative embodiment.

FIG. 12 is a flow chart illustrating a usage inference process in an illustrative embodiment.

FIG. 13 is a diagram illustrating the usage inference process for a patient in an illustrative embodiment.

FIGS. 14A-14B are flow charts illustrating methods of estimating a time gap in illustrative embodiments.

FIG. 15 is a flow chart illustrating a method of estimating a usage duration in an illustrative embodiment.

FIG. 16 is a flow chart illustrating a method of processing cohort criteria in an illustrative embodiment.

FIG. 17 is a flow chart illustrating a method of genetic criteria processing in an illustrative embodiment.

FIG. 18 is a block diagram illustrating data analysis resources of a genomic data system in an illustrative embodiment.

FIGS. 19-21 illustrate a medication usage GUI in an illustrative embodiment.

### DETAILED DESCRIPTION

The figures and the following description illustrate specific exemplary embodiments. It will thus be appreciated that those skilled in the art will be able to devise various arrangements that, although not explicitly described or shown herein, embody the principles of the embodiments and are included within the scope of the embodiments. Furthermore, any examples described herein are intended to aid in understanding the principles of the embodiments, and are to be construed as being without limitation to such specifically recited examples and conditions. As a result, the inventive concept(s) is not limited to the specific embodiments or examples described below, but by the claims and their equivalents.

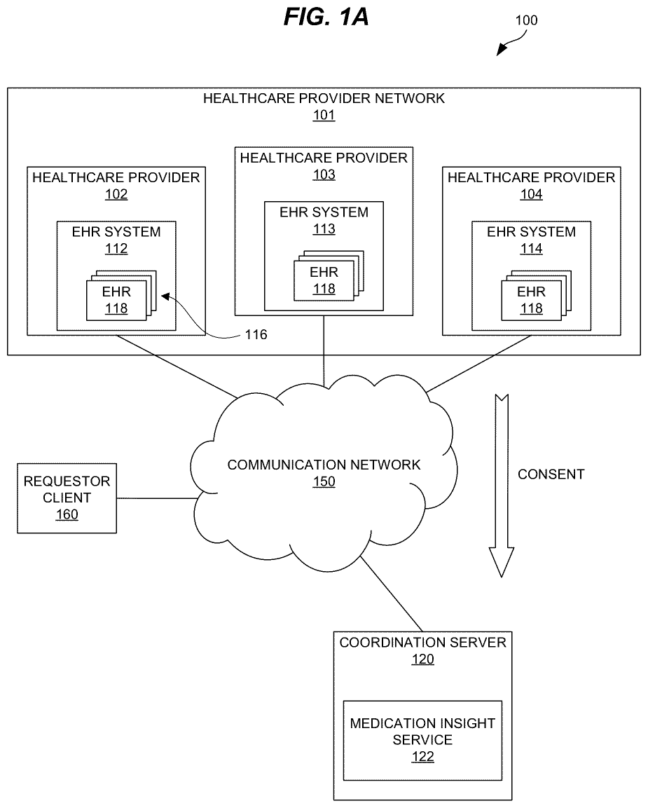

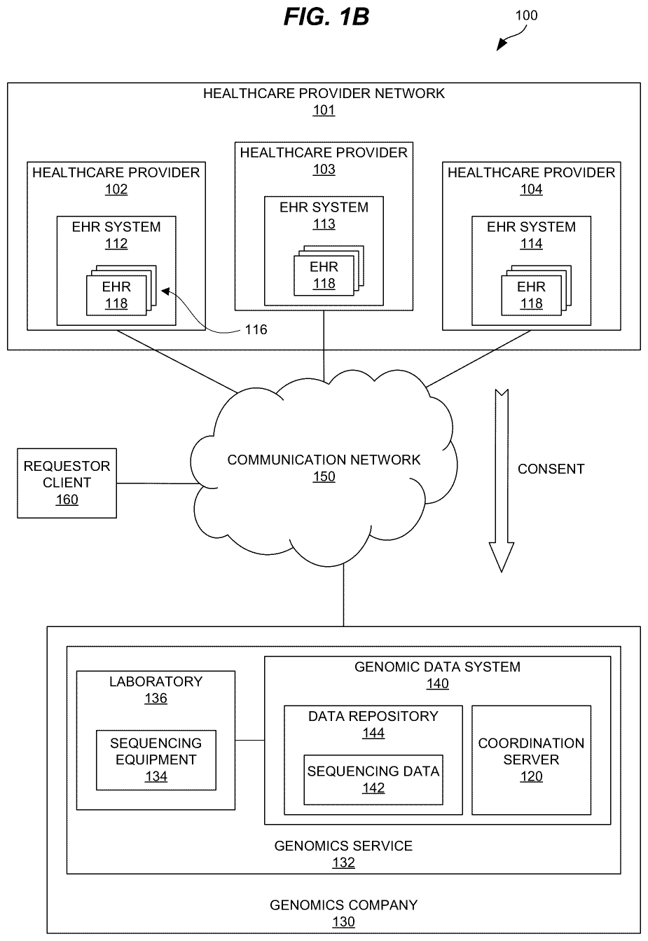

FIG. 1A is a block diagram of a medication insight architecture 100 in an illustrative embodiment. At a high level, medication insight architecture 100 comprises any combination of systems, components, and/or devices configured to compile and/or analyze health-related data, including data regarding medications or prescription drugs. In an embodiment, medication insight architecture 100 includes one or more EHR systems 112-114 (also referred to as EHR servers) of one or more healthcare providers 102-104 belonging to a healthcare provider network 101. A healthcare provider 102-104 is a licensed person or organization that provides healthcare services. A healthcare provider 102-104 implements an EHR system 112-114 that maintains, tracks, and/or stores EHR data 116 for a plurality of patients. An Electronic Health Record (EHR) 118 is an electronic or digital version of a patient's medical history maintained by a healthcare provider or the like. EHR data 116 (also referred to generally as an EHR(s) 118) may include patient care information, including demographics (e.g., date of birth or age, gender, ethnicity, blood type, income, postal code, etc.), progress notes, problems, medications/prescriptions, vital signs, past medical history, immunizations, laboratory data or test results, radiology reports, medical codes (e.g., International Classification of Diseases (ICD) codes in an ICD-10 format, Current Procedural Terminology (CPT) codes, etc.), and/or other information. An EHR system 112-114 may be implemented on a cloud-based or cloud-computing platform, and/or may be implemented on a hardware or server-based platform on-site for the healthcare provider 102-104. Although one healthcare provider network 101 is shown, medication insight architecture 100 may include multiple healthcare provider networks 101 each comprising one or more EHR systems 112-114.

Medication insight architecture 100 further includes a coordination server 120, which is a data processing apparatus configured to gather, analyze, or process EHR data 116 for patients, or otherwise support medication research or extract information/insights from EHR data 116. Coordination server 120 may be configured to provide a medication insight service 122, which in general, has access to one or more databases of healthcare information, such as EHR data 116 maintained by one or more EHR systems 112-114. The medication insight service 122 may be a fee-based service, such as a subscription-based service where a subscription is obtained to receive the medication insight service 122, a transaction-based service where a fee is charged per request or transaction, etc. As illustrated in FIG. 1A, coordination server 120 may have consent to access the EHR data 116 maintained by EHR systems 112-114, and/or other healthcare information. In response to a request, such as from a requestor client 160, the medication insight service 122 identifies a particular medication of interest, selects or identifies a cohort of patients that matches certain criteria, and compiles EHR data 116 for the cohort. The medication insight service 122 may also perform certain analysis of the EHR data 116 for the cohort.

Coordination server 120 is configured to communicate with external systems or devices via a communication network 150. Communication network 150 may comprise a Wide Area Network (WAN), such as the Internet, a telecommunications network, an enterprise network or private network, a Wireless Local Area Network (WLAN), etc., or any combination thereof. As will be described in more detail below, coordination server 120 is configured to receive or retrieve EHR data 116 stored in one or more EHR systems 112-114, via communication network 150. Coordination server 120 is further configured to communicate with a requestor client 160 and/or other external systems not shown, via communication network 150.

FIG. 1B is a block diagram of a medication insight architecture 100 in another illustrative embodiment. In this embodiment, coordination server 120 may be implemented in or associated with a genomics service 132 offered by a genomics company 130. Genomics service 132 is in the field of bioinformatics, which is a scientific field related to the development or application of tools or applications to analyze and interpret biological data, such as DNA (deoxyribonucleic acid) sequences. At a high level, genomics service 132 comprises collection, storage, and/or analysis of genomic or genetic data. For the genomics service 132, genomics company 130 may perform or offer sample collection, DNA (deoxyribonucleic acid) or genomic sequencing, secure data storage of the sequencing data generated by sequencing processes, analysis of the sequencing data, etc. The genomics service 132 may be a fee-based service, such as a subscription-based service where a subscription is obtained to receive the genomics service 132.

For a sequencing process, genomics company 130 may implement or use sequencing equipment 134 (e.g., a sequencing instrument(s), a sequencing platform, a next-generation sequencing (NGS) platform, etc.) at a laboratory 136 or the like, which is configured to perform a sequencing process on biological samples. For example, DNA sequencing is a process of determining an exact sequence of nucleotides, or bases, in a DNA molecule. Sequencing equipment 134 may therefore include a DNA sequencer and/or other instruments configured to determine the order of the four bases: G (guanine), C (cytosine), A (adenine), and T (thymine). Genomic sequencing is a process of determining the entire genetic makeup of an organism.

Genomics company 130 may further implement a genomic data system 140 configured to store (i.e., secure data storage) sequencing data 142 (also referred to as genomic sequencing data or genetic sequencing data) in a data repository 144, analyze sequencing data 142, and/or otherwise manage sequencing data 142. For example, genomic data system 140 may process the sequencing data 142 (e.g., raw sequence data) to identify variants or alleles (i.e., variant calling). The sequencing data 142 as described herein may include raw DNA or genomic sequences (e.g., order of the bases), and any associated data extracted from the raw sequences, such as aligned sequence data, variant information or variant call data, etc. Genomic data system 140 may be implemented at a laboratory 136 of the genomics company 130, such as on servers or other on-premises resources at the laboratory 136. Alternatively, genomic data system 140 may be implemented on one or more external platforms, such as a cloud infrastructure of a cloud computing platform. Cloud computing is the delivery of computing resources, including storage, processing power, databases, networking, analytics, artificial intelligence, and software applications, over an internet connection. Some examples of a cloud computing platform may comprise Amazon Web Services (AWS), Google Cloud, Microsoft Azure, etc. Further, although genomics company 130 is illustrated as implementing sequencing equipment 134 and genomic data system 140, it is understood that the sequencing equipment 134 and genomic data system 140 may be distributed among different companies, entities, platforms, etc.

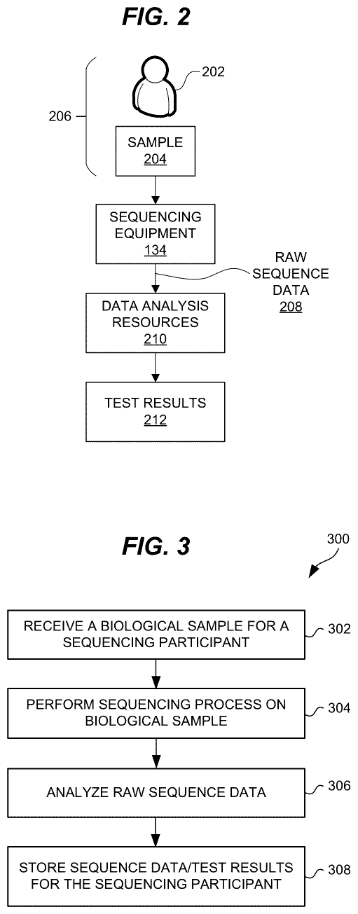

FIG. 2 is a block diagram illustrating genetic testing in an illustrative embodiment. FIG. 3 is a flow chart illustrating a method 300 of genetic testing in an illustrative embodiment. The steps of the flow charts described herein are not all inclusive and may include other steps not shown, and the steps may be performed in an alternative order. A biological sample 204 (e.g., blood, saliva, etc.) of an individual 202 is received at laboratory 136 for sequencing (step 302). An individual 202 that volunteers or consents to genomic sequencing of a biological sample 204 is referred to as a sequencing participant 206. The sequencing equipment 134 at laboratory 136 performs a sequencing process on the biological sample 204 to generate raw sequence data 208 associated with the sequencing participant 206 (step 304). Data analysis resources 210 may then analyze or otherwise process the raw sequence data 208, such as alignment, variant calling, and/or any other analysis (step 306). The analysis process generates test results 212 (also referred to as diagnostic results, analysis results, genomic analysis results, analysis output, etc.). The raw sequence data 208 and any data or information generated by the analysis of the raw sequence data 208, such as the aligned sequence data, variant information or variant call data, etc., may be collectively referred to as sequencing data 142 for, or associated with, a sequencing participant 206. The sequencing data 142 may comprise data for a whole genome, a subset of the genes that make up a genome, etc. The sequencing data 142 and/or test results 212 are stored in secure data storage (step 308), such as in a data repository.

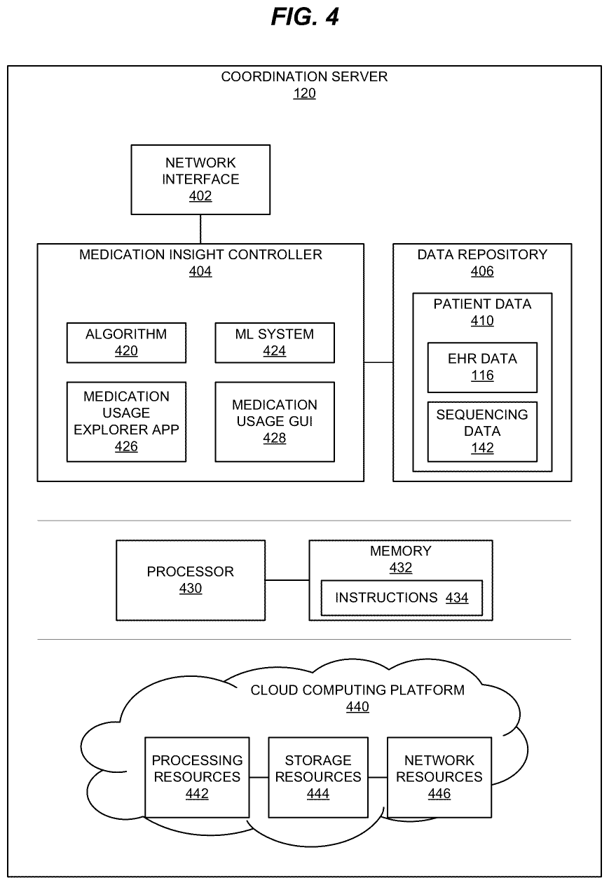

FIG. 4 is a block diagram of coordination server 120 in an illustrative embodiment. Coordination server 120 may include the following subsystems: a network interface component 402, a medication insight controller 404, and a data repository 406 that operate on one or more platforms. Network interface component 402 may comprise circuitry, logic, hardware, means, etc., configured to exchange messages, documents, and/or electronic data communications with external devices or systems. Network interface component 402 may operate using a variety of protocols and/or Application Programming Interfaces (APIs). Medication insight controller 404 may comprise circuitry, logic, hardware, means, etc., configured to process EHR data 116, sequencing data 142, and/or other patient data 410 or health-related data to select or identify a cohort of patients, analyze EHR data 116 for a cohort, and/or perform other functions. Medication insight controller 404 may execute one or more algorithms 420, one or more machine learning (ML) systems 424, etc., to perform one or more actions or tasks as described herein. Medication insight controller 404 may execute a medication usage explorer application 426 to perform the functions or operations described below. Medication insight controller 404 may also provide a medication usage Graphical User Interface (GUI) 428, which is a digital interface configured to interact with a user. Data repository 406 comprises secure data storage configured to store EHR data 116, sequencing data 142, and/or other patient data 410.

One or more of the subsystems of coordination server 120 may be implemented on a hardware platform comprised of analog and/or digital circuitry. For example, network interface component 402 and/or medication insight controller 404 may be implemented on one or more processors 430 that execute instructions 434 (i.e., computer readable code) for software that are loaded into memory 432. A processor 430 comprises an integrated hardware circuit configured to execute instructions 434 to provide the functions of coordination server 120. Processor 430 may comprise a set of one or more processors or may comprise a multi-processor core, depending on the particular implementation. Memory 432 is a non-transitory computer readable storage medium for data, instructions, applications, etc., and is accessible by processor 430. Memory 432 is a hardware storage device capable of storing information on a temporary basis and/or a permanent basis. Memory 432 may comprise a random-access memory, or any other volatile or non-volatile storage device.

One or more of the subsystems of coordination server 120 may be implemented on cloud computing platform 440 (e.g., AWS) or another type of processing platform. Cloud resources may be provisioned on cloud computing platform 440, such as processing resources 442 (e.g., physical or hardware processors, a server, a virtual server or virtual machine (VM), a virtual central processing unit (vCPU), etc.), storage resources 444 (e.g., physical or hardware storage, virtual storage, etc.), and/or networking resources 446, although other resources are considered herein. Coordination server 120 may be built upon the provisioned resources with instructions, programming, code, etc. For example, network interface component 402 may be provisioned on networking resources 446, medication insight controller 404 may be provisioned on processing resources 442, and data repository 406 may be provisioned on storage resources 444.

Coordination server 120 may include various other components not specifically illustrated in FIG. 4.

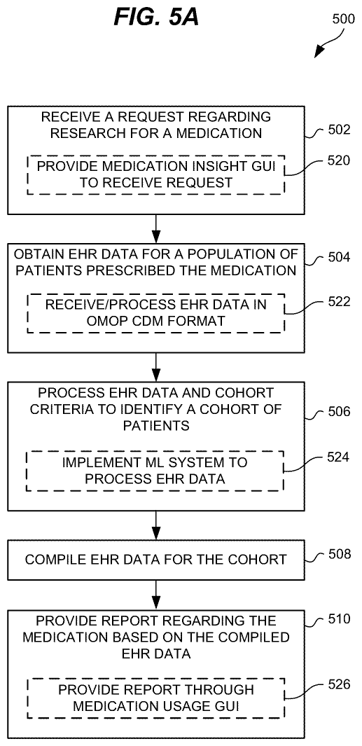

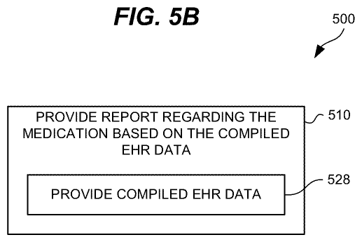

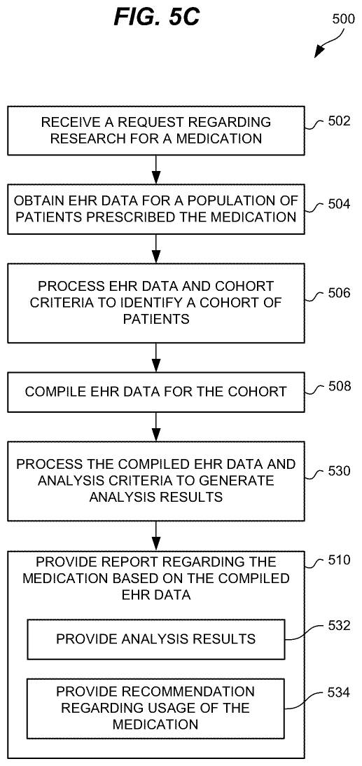

FIGS. 5A-5C are flow charts illustrating a method 500 of processing EHR data 116 for patients in an illustrative embodiment. The steps of method 500 will be described with reference to coordination server 120 in FIG. 4, but those skilled in the art will appreciate that method 500 may be performed in other systems or devices.

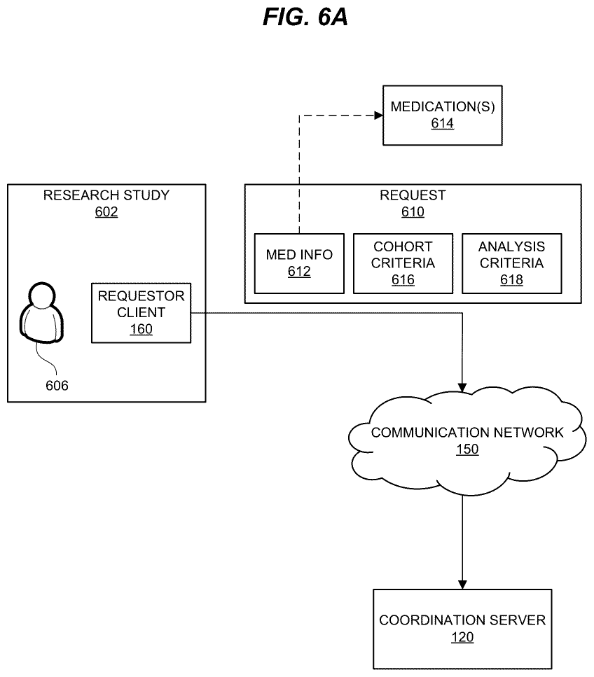

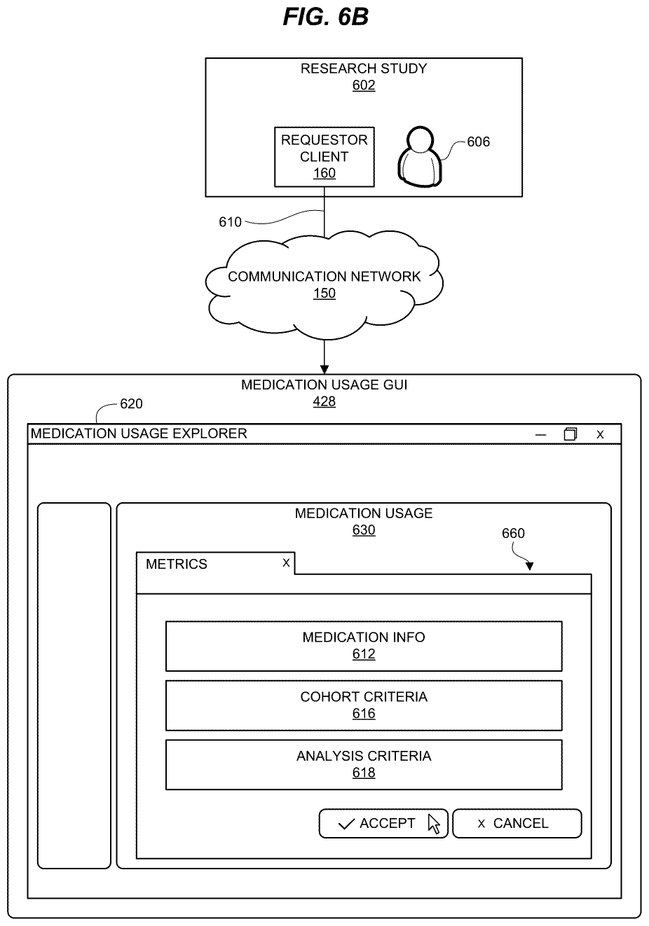

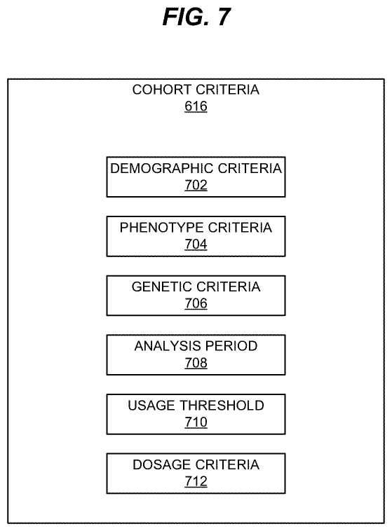

In FIG. 5A, medication insight controller 404 (e.g., through network interface component 402 via the communication network 150) receives a request regarding research, a study, insights, etc., for a medication of interest (step 502). FIGS. 6A-6B are diagrams illustrating a request received by coordination server 120 in an illustrative embodiment. In FIG. 6A, a requestor 606 may use a requestor client 160 (e.g., a personal computer (PC), laptop, smartphone, etc.) to submit a request 610 to coordination server 120. The request 610 is for analysis, study, investigation, examination, or drug-related research regarding one or more medications 614. For example, requestor 606 may be involved in a research study 602 regarding a medication 614, such as an observational study, a clinical trial (e.g., a type of research study that tests medical, surgical, or behavioral intervention in individuals), etc. The request 610 may include or indicate medication information 612 (e.g., one or more medication identifiers (ID)) that specifies a medication or medications of interest. The request 610 may include or indicate cohort criteria 616 to define a cohort from a population of patients. Cohort criteria 616 comprises characteristics, parameters, attributes, conditions, or other information to define a cohort from a population of patients. FIG. 7 is a diagram illustrating cohort criteria 616 in an illustrative embodiment. For example, the cohort criteria 616 may include demographic criteria 702 (e.g., age, gender, ethnicity, etc.), trait or phenotype criteria 704 (e.g., height, weight, Body Mass Index (BMI), blood pressure, pulse), genetic criteria 706 (e.g., genes of interest, chromosome position, one or more variants and/or variant type (deletion, duplication, indel, insertion, single nucleotide, etc.)), an analysis period 708 indicating a desired or target time period of usage for a medication, a usage threshold 710 (i.e., a minimum usage duration of a medication), dosage criteria 712 regarding a medication, etc. The cohort criteria 616 may include other information as desired, such as health conditions (e.g., disease, diagnosis, symptom, etc.), symptoms of interest, lab results, comorbidities, etc.

As in FIG. 6A, the request 610 may include or indicate analysis criteria 618 for analyzing EHR data 116 for the cohort. For example, the analysis criteria 618 may specify analysis of weight loss for patients of the cohort, analysis of cardiovascular events or disorders for patients of the cohort, analysis of symptoms or side-effects (e.g., chest pain, nausea, drowsiness, etc.) experienced by patients of the cohort, etc.

In an embodiment, medication insight controller 404 may provide or generate a medication usage GUI 428 configured to receive the request 610 from the requestor 606 (optional step 520 of FIG. 5A). In FIG. 6B, medication usage GUI 428 provides a medication usage explorer 620, which is a window, webpage, portal, dashboard, etc., that allows a requestor 606 to interact with medication insight controller 404/medication usage explorer application 426 via communication network 150. Medication usage explorer 620 may display one or more graphical elements to interact with a user. In this example, medication usage explorer 620 may display a medication usage graphical element 630 positioned or located at a display portion 660. A display portion is a section, segment, or part occupying an area or partial area of a GUI. Medication usage graphical element 630 is an interactive graphical element configured to allow a requestor 606 to enter or input medication information 612 (e.g., one or more medication IDs), cohort criteria 616, analysis criteria 618, and/or other information. In the example in FIG. 6B, medication usage explorer 620 provides a requestor 606 remote access through requestor client 160 over communication network 150. Although not shown in FIG. 6B, requestor 606 may have to set up an account with the provider of the medication usage explorer application 426 prior to accessing medication usage explorer 620, as the medication usage explorer application 426 may provide a paid-for or subscription service. Also, requestor 606 may be authenticated by medication usage explorer application 426 through a login form/page prior to accessing medication usage explorer 620.

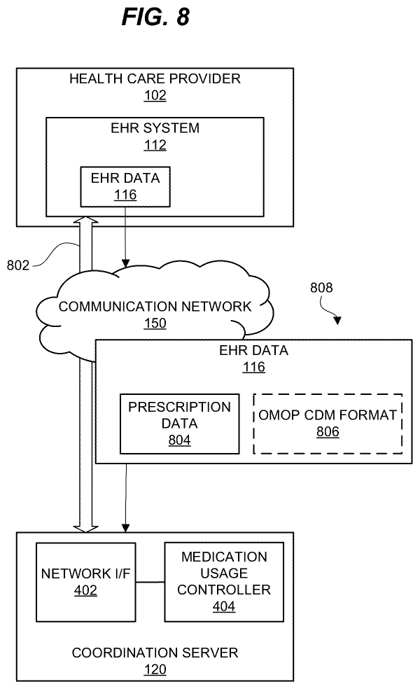

In FIG. 5A, medication insight controller 404 obtains, collects, gathers, or acquires EHR data 116 for a population of patients prescribed the medication 614 (step 504). In an embodiment, the EHR data 116 is anonymized before reaching the coordination server 120. FIG. 8 is a diagram illustrating retrieval of EHR data 116 in an illustrative embodiment. Medication insight controller 404 retrieves or collects EHR data 116 comprising prescription data 804 for a medication 614 of interest. Medication insight controller 404 may establish a secure connection 802 with one or more EHR systems 112-114 over communication network 150 via network interface component 402. Medication insight controller 404 may then receive the EHR data 116 for one or more patients from the EHR system(s) 112-114 (e.g., pushed by the EHR system(s) 112-114 or pulled from the EHR system(s) 112-114 using a request/response model) via the secure connection 802. For example, medication insight controller 404 may collect or ingest (periodically) the EHR data 116 in batches 808 from the EHR system(s) 112-114, and filter the EHR data 116 based on the medication 614 of interest. A batch 808 of EHR data 116 may comprise a massive or voluminous amount of EHRs 118 for patients, such as in excess of one hundred thousand records, one million records, etc. Medication insight controller 404 may filter through the voluminous amount of EHRs 118 to identify the records for patients prescribed the medication 614 of interest.

In one embodiment, when medication insight controller 404 is implemented in a genomic data system 140, genomic data system 140 may receive a biological sample 204 for an individual 202 from a healthcare provider 102-104 for sequencing at laboratory 136, and the EHR system 112 of the healthcare provider 102-104 may send the EHR data 116 of the individual 202 to genomic data system 140 when sending the biological sample 204 for sequencing (assuming authorizations are provided by the individual 202).

In an embodiment, coordination server 120 may receive and/or process the EHR data 116 in Observational Medical Outcomes Partnership (OMOP) Common Data Model (CDM) format 806 (optional step 522 of FIG. 5A). OMOP CDM is a standard designed to standardize the structure and content of observational data, such as EHR data 116. Medication insight controller 404 is therefore configured to process the EHR data 116 in OMOP CDM format 806, such as to identify tables/fields that contain prescription data 804, demographic information, phenotype information, measurement or observation information, notes, etc.

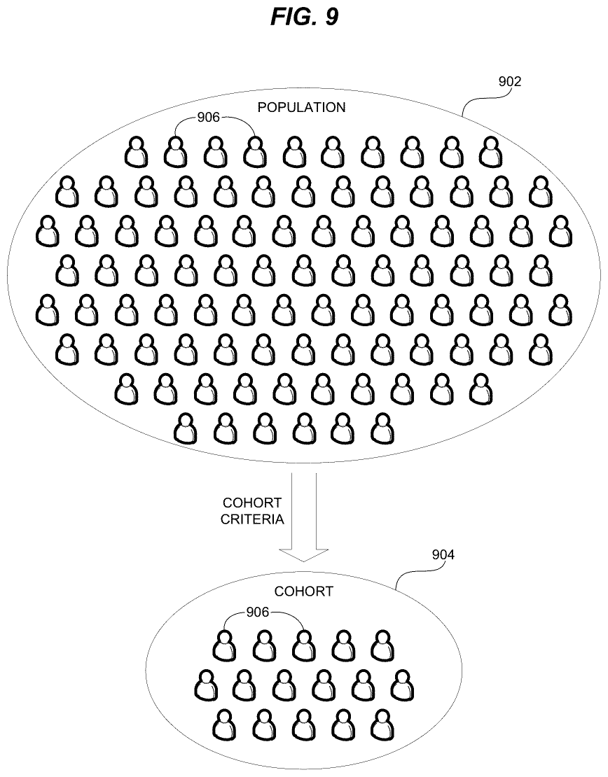

In FIG. 5A, medication insight controller 404 constructs or assembles a cohort of patients based on the EHR data 116 and the cohort criteria 616. To do so, medication insight controller 404 performs electronic data processing on the EHR data 116 for the population of patients based on the cohort criteria 616 to identify a cohort of patients from the population (step 506). In other words, medication insight controller 404 may parse or search (e.g., automatically in response to the input from the requestor 606) the EHR data 116 to identify EHR data 116 that matches the cohort criteria 616. FIG. 9 illustrates identification of a cohort from a population of patients in an illustrative embodiment. Data repository 406 stores EHR data 116 for a population 902 of patients 906. The population 902 of patients 906 may be an aggregation of multiple healthcare providers 102-104, for an individual healthcare provider 102-104, etc., where each of the patients 906 was prescribed the mediation(s) 614 of interest. Medication insight controller 404 performs electronic data processing on the EHR data 116 for the population 902 based on the cohort criteria 616 to identify a cohort 904 of patients 906 from the population 902. Cohort 904 is a group of patients 906 (i.e., humans) with one or more shared characteristics in the EHR data 116 or other data, based on the cohort criteria 616.

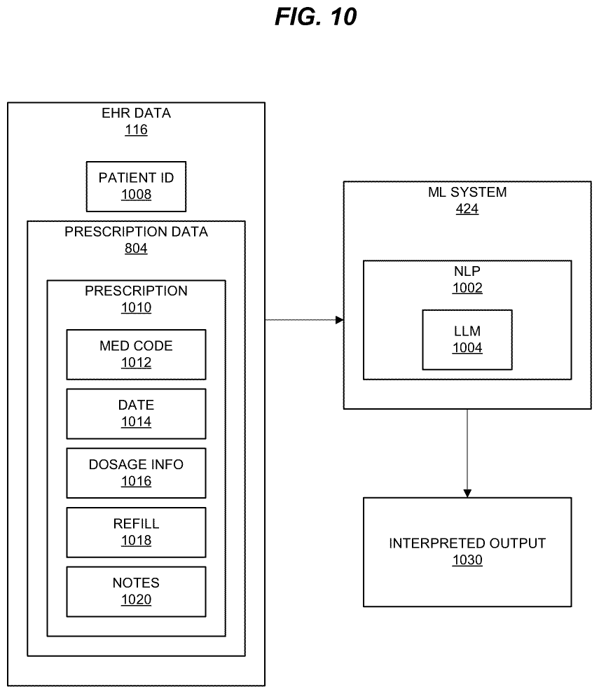

In an embodiment, medication insight controller 404 may implement a ML system 424 to process the EHR data 116 (optional step 524 of FIG. 5A). FIG. 10 is a diagram illustrating use of an ML system 424 to process the EHR data 116 in an illustrative embodiment. ML system 424 may be trained to interpret human language. Some examples of ML system 424 are a Natural Language Processing (NLP) model 1002, a Large Language Model (LLM) 1004, etc. Thus, ML system 424 may be implemented to interpret the EHR data 116, and generate interpreted output 1030. In an example, each EHR 118 is associated with a specific (e.g., anonymized) patient identifier (ID) 1008 of a patient 906. The prescription data 804 of an EHR 118 indicates one or more prescriptions 1010 written by a physician or healthcare practitioner for the patient 906. For example, a prescription 1010 may include a medication name and/or medication code 1012, a prescribed date 1014 (i.e., date the prescription was written), dosage information 1016, refill information 1018 (i.e., number of refills), notes 1020, etc. The dosage information 1016 may indicate a dose (i.e., the amount of drug taken at any one time), a dosage regimen (i.e., the frequency at which the drug doses are given), a prescription time period (i.e., a number of days for taking the drug), etc. Prescription data 804 may vary in its format. For example, prescription data 804 or a portion thereof may be in plain text as written by a physician. Medication insight controller 404 may therefore input prescription data 804 into the ML system 424 to interpret the prescription data 804. One technical benefit is the ML system 424 generates a standardized output (i.e., the interpreted output 1030) of the prescription data 804 across different EHRs, different physicians, etc.

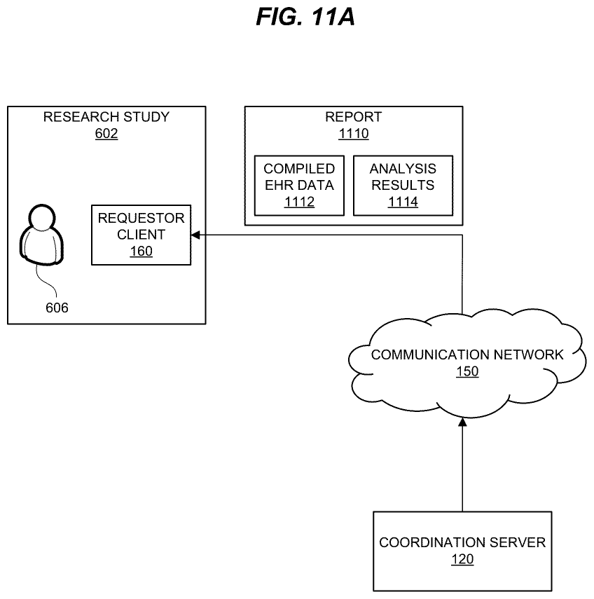

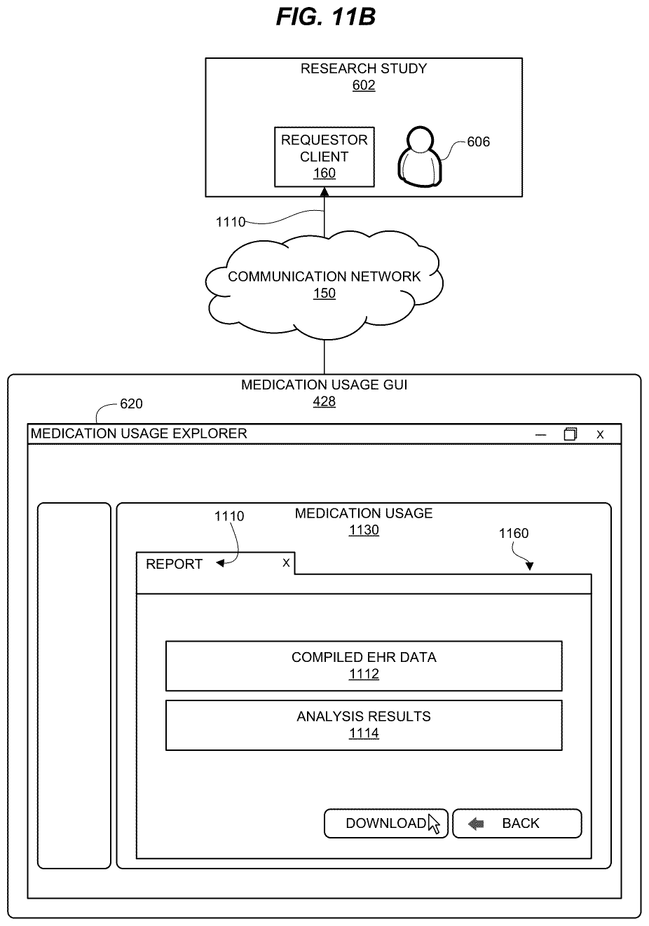

In FIG. 5A, medication insight controller 404 compiles the EHR data 116 for the cohort 904 of patients 906 (step 508). For example, medication insight controller 404 may create a research file (e.g., a. zip file) that includes the compiled EHR data for the patients 906 of the cohort 904, may create a directory that stores the compiled EHR data for the patients 906 of the cohort 904, etc. Medication insight controller 404 then provides a report, summary, description, or representation regarding the medication 614 of interest based on the compiled EHR data (step 510). FIGS. 11A-11B are diagrams illustrating coordination server 120 providing a report 1110 regarding the medication 614 in an illustrative embodiment. In FIG. 11A, medication insight controller 404 (through network interface component 402) may transmit, send, or otherwise provide the report 1110 to requestor client 160 in response to the request 610. For example, medication insight controller 404 may send an email, an FTP (File Transfer Protocol or secure FTP) message, etc., containing the report 1110, may provide a link to the report 1110 (e.g., a Uniform Resource Locator (URL)), etc. In an embodiment, the report 1110 may comprise the compiled EHR data 1112 for the patients 906 of the cohort 904 (see optional step 528 of FIG. 5B). In an embodiment, the report 1110 may comprise analysis results 1114 resulting from analysis of the compiled EHR data 1112 (see FIG. 11A), which is described in further detail below.

In an embodiment, coordination server 120 may provide the report 1110 through medication usage GUI 428 (optional step 526 of FIG. 5A). In FIG. 11B, medication usage explorer 620 may display a medication usage graphical element 1130 positioned or located at a display portion 1160. Medication usage graphical element 1130 is an interactive graphical element configured to display the report 1110, allow a requestor 606 to download or otherwise access the report 1110, etc. One technical benefit is medication usage explorer 620 provides the requestor 606 remote access to the compiled EHR data 1112 and/or the analysis results 1114 resulting from analysis of the compiled EHR data 1112.

In identifying the cohort 904, medication insight controller 404 processes the prescription data 804 for the patients 906 regarding prescriptions 1010 of the medication 614. However, the prescription data 804 may be incomplete within EHR data 116 regarding usage of a medication 614, the prescription data 804 may be untrustworthy or inaccurate, etc. For example, although a prescription 1010 may include a prescribed date 1014 indicating when the medication 614 was prescribed and/or ordered, this date is not necessarily the date that the patient 906 picks up the medication 614 or initiates use of the medication 614. Further, prescription data 804 within an EHR 118 rarely includes a stop date of a medication and, when present, is often not an accurate stop date. It is not uncommon for a patient 906 to start using a prescribed medication, and then stop use of the medication (e.g., owing to an allergic reaction, side effects, inefficacy, or other reasons) without this halt in medication use being reported in an EHR 118. Thus, it is a challenge to determine which patients 906 to include in the cohort 904 relating to use of a medication 614, even when the patient 906 has received a prescription 1010 for the medication 614.

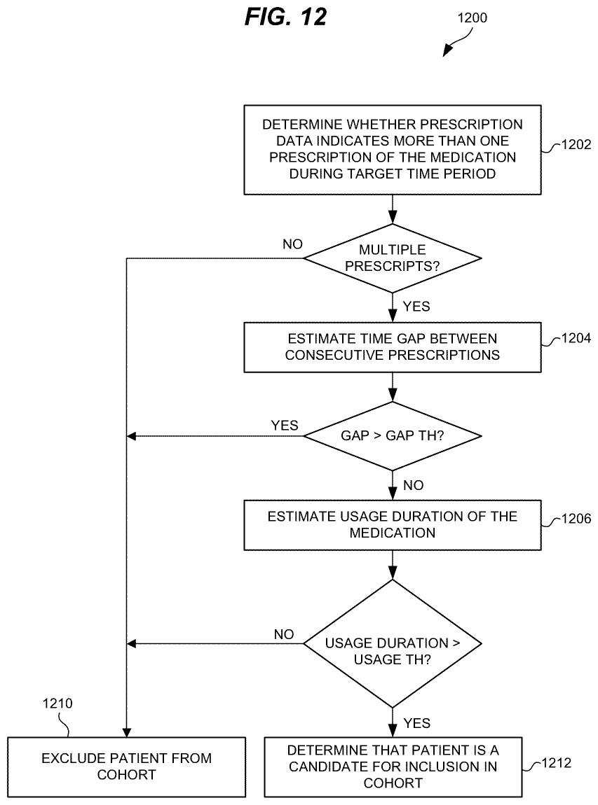

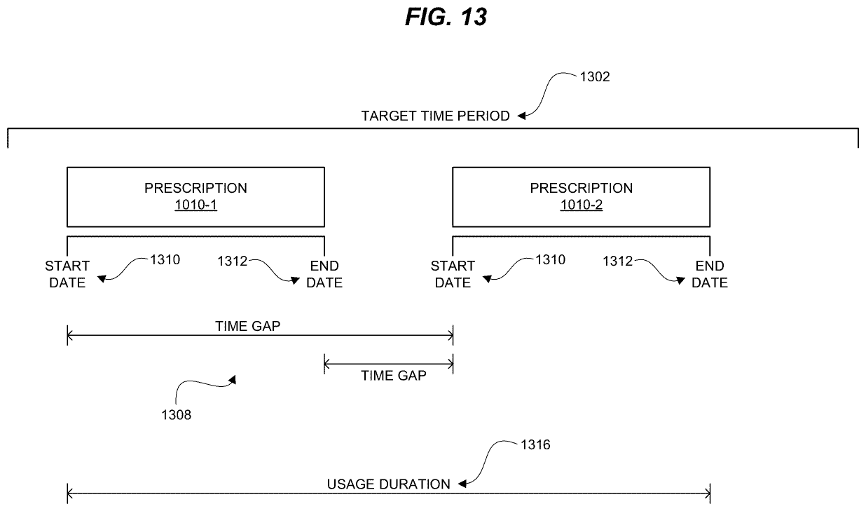

In an embodiment, medication insight controller 404 may implement a usage inference process to estimate the medication usage of a patient 906 based on the prescription data 804. Medication insight controller 404 then uses the estimated medication usage for a patient 906 to determine whether to include or exclude the patient 906 from the cohort 904. FIG. 12 is a flow chart illustrating a usage inference process 1200 in an illustrative embodiment. It is noted that the order of the steps shown for the usage inference process 1200 in FIG. 12 is provided as an example, and the steps may be performed in an alternative order. FIG. 13 is a diagram illustrating the usage inference process 1200 for a patient 906 in an illustrative embodiment.

The usage inference process 1200 looks for patterns of repeated or ongoing use of a medication 614 or chain of prescriptions 1010 over a target time period 1302. The target time period 1302 (see FIG. 13) may be all time up to the present, a past number of days, months, or years (e.g., six months, one year, ten years, etc.), a specific window of time (e.g., 2001-2011), etc. The target time period 1302 may be referred to as the “analysis period”, and may be predefined, may be specified in the cohort criteria 616, etc. Medication insight controller 404 processes the prescription data 804 to determine whether the prescription data 804 indicates more than one prescription 1010 of the medication 614 during the target time period 1302 (step 1202 in FIG. 12). One technical benefit of determining whether the prescription data 804 indicates multiple prescriptions 1010 is to exclude patients 906 who likely took the medication 614 of interest for a relatively brief period of time with no evidence of longer-term use. There are several reasons why a repeat prescription 1010 may not be present, each likely representing drug non-compliance or discontinuation. After a prescription 1010 is first written, it may not have been filled by a pharmacy, the medication 614 may not have been picked up by the patient 906, the patient 906 may have halted use of the medication 614 owing to side effects, adherence challenges, etc. Medication insight controller 404 may then exclude a patient 906 from the cohort 904 when the prescription data 804 indicates less than multiple prescriptions 1010 during the target time period 1302 (step 1210 of FIG. 12). One technical benefit is medication insight controller 404 is able to selectively exclude patients 906 that may have quickly discontinued use after an initial prescription, failed to pick up their medication, etc. Excluding patients 906 in this manner maximizes “sensitivity” in identification of the medication-using cohort 904, at the potential cost of specificity (i.e., there may be patients 906 who continued use of the medication 614 for some time even though they were only prescribed one prescription). In FIG. 13, multiple prescriptions 1010 (i.e., prescriptions 1010-1 and 1010-2) are prescribed during the target time period 1302, so this patient 906 is a candidate for inclusion in the cohort 904.

The usage inference process 1200 also looks for patterns of continued use of the medication 614 over the target time period 1302. The usage inference process 1200 may therefore determine whether there are large gaps between prescriptions 1010. Thus, when the prescription data 804 indicates multiple prescriptions 1010, medication insight controller 404 processes the prescription data 804 to estimate a time gap 1308 between consecutive prescriptions 1010 (step 1204 of FIG. 12). Medication insight controller 404 may then exclude the patient 906 from the cohort 904 when a time gap 1308 between consecutive prescriptions 1010 is greater than a gap threshold (step 1210 of FIG. 12). The gap threshold may be predefined, may be specified in the cohort criteria 616, etc. For example, the gap threshold may be two weeks, one month, two months, six months, etc. In some embodiments, the gap threshold corresponds with an amount of time that a prescription 1010 is expected to last for a patient when a medication 614 for the prescription 1010 is regularly used by the patient. FIG. 13 illustrates a time gap 1308 estimated between prescription 1010-1 and prescription 1010-2, and the time gap 1308 is compared to the gap threshold to determine whether the patient 906 has a pattern of continued use. Although two prescriptions 1010 are illustrated in FIG. 13, medication insight controller 404 may estimate time gaps 1308 between any two consecutive prescriptions 1010. One technical benefit is patients 906 that discontinue use of the medication 614 for extended periods of time may be excluded from the cohort 904.

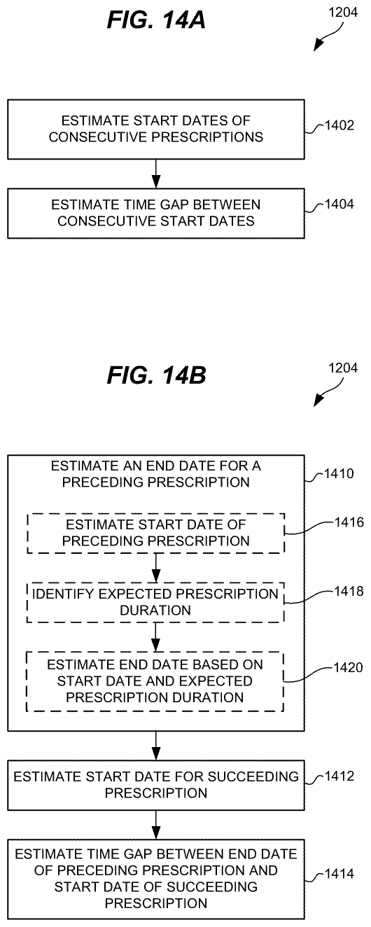

FIGS. 14A-14B are flow charts illustrating methods of estimating a time gap 1308 in illustrative embodiments. In FIG. 14A, to estimate a time gap 1308 between consecutive prescriptions 1010 in one embodiment, medication insight controller 404 estimates a start date 1310 for a preceding prescription 1010-1 (in time order) and a start date 1310 for a succeeding prescription 1010-2 (step 1402). The start date 1310 for a prescription 1010 may be estimated as the prescribed date 1014 or order date indicated in the prescription data 804. Medication insight controller 404 may then estimate the time gap 1308 between consecutive start dates 1310 (step 1404).

In FIG. 14B, to estimate a time gap 1308 between consecutive prescriptions 1010 in another embodiment, medication insight controller 404 estimates an end date 1312 for a preceding prescription 1010-1 (step 1410), and estimates a start date 1310 for a succeeding prescription 1010-2 (step 1412). It is common for a prescription 1010 to include a prescribed date 1014, so medication insight controller 404 may estimate the start date 1310 based on the prescribed date 1014. However, it is common for the prescription data 804 to be devoid of an end date 1312 for a prescription 1010. When the prescription data 804 includes or indicates an end date 1312, medication insight controller 404 may estimate the end date 1312 as the actual end date. When the prescription data 804 does not include an end date 1312 or an end date 1312 is considered untrustworthy (e.g., as indicated by a parameter indicating that end dates are to be ignored, as indicated by an end date followed another prescription for the same medication, etc.), medication insight controller 404 may estimate a start date 1310 for the preceding prescription 1010-1 (optional step 1416), and identify an expected prescription duration (e.g., one month, three months, etc.) of a prescription 1010 for the medication 614 (optional step 1418). The expected prescription duration may be predefined, and may be based on an average prescription duration for a medication 614, input or recommendation from a drug manufacturer, input or recommendation from a medical practitioner, etc. Medication insight controller 404 may then estimate the end date 1312 of the preceding prescription 1010-1 as the start date 1310 plus the expected prescription duration (optional step 1420). For example, if the start date 1310 is January 1 and the expected prescription duration is three months, then medication insight controller 404 may estimate the end date 1312 as April 1. Medication insight controller 404 may then estimate the time gap 1308 between the end date 1312 of the preceding prescription 1010-1 and the start date 1310 of the succeeding prescription 1010-2 (step 1414).

The usage inference process 1200 also determines how long there was continued use of the medication 614 over the target time period 1302. Thus, medication insight controller 404 processes the prescription data 804 to estimate a usage duration 1316 of the medication 614 during the target time period 1302 (step 1206 of FIG. 12). The usage duration 1316 indicates how long a patient 906 used a medication 614 “continuously” (i.e., without large gaps). Thus, the usage duration 1316 is across multiple prescriptions 1010 where the time gap 1308 between consecutive prescriptions 1010 is less than the gap threshold. Medication insight controller 404 may then exclude the patient 906 from the cohort 904 when the usage duration 1316 is less than a usage threshold (step 1210 of FIG. 12). The usage duration 1316 may be predefined, may be specified in the cohort criteria 616, etc. For example, the usage duration 1316 may be six months, one year, etc. One technical benefit is patients 906 that have insufficient use of the medication 614 may be excluded from the cohort 904, as effects of the medication 614 may not be evident.

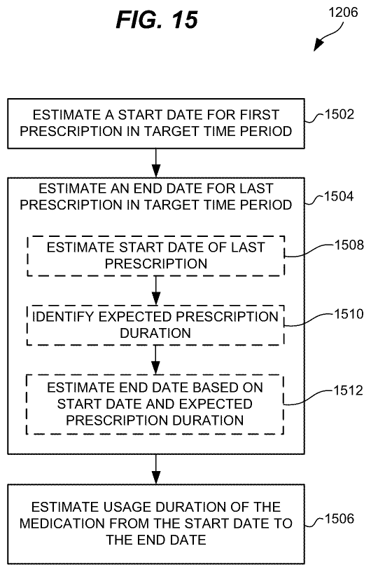

FIG. 15 is a flow chart illustrating a method of estimating a usage duration 1316 in an illustrative embodiment. To estimate a usage duration 1316 in one embodiment, medication insight controller 404 may estimate a start date 1310 for the first prescription 1010-1 in the target time period 1302 (step 1502). As described above, the start date 1310 for a prescription 1010 may be estimated as the prescribed date 1014 or order date indicated in the prescription data 804. Medication insight controller 404 may estimate an end date 1312 for the last prescription 1010-2 in the target time period 1302 (step 1504). As described above, when the prescription data 804 includes or indicates an end date 1312 for the last prescription 1010-2, medication insight controller 404 may estimate the end date 1312 as the actual end date. When the prescription data 804 does not include an end date 1312 or an end date 1312 is considered untrustworthy, medication insight controller 404 may estimate a start date 1310 for the last prescription 1010-2 (optional step 1508), identify an expected prescription duration of a prescription 1010 for the medication 614 (optional step 1510), and estimate the end date 1312 of the last prescription 1010-2 as the start date 1310 plus the expected prescription duration (optional step 1512). When the last prescription 1010-2 is current or active, the end date 1312 may be estimated as the end date of the target time period 1302. Medication insight controller 404 may then estimate the usage duration 1316 between the start date 1310 of the first prescription 1010-1 and the end date 1312 of the last prescription 1010-2 (step 1506).

When the prescription data 804 for a patient 906 satisfies the conditions of the usage inference process 1200 as in FIG. 12, medication insight controller 404 may determine that the patient 906 is a candidate for inclusion in the cohort 904 (step 1212), as there is sufficient evidence to infer ongoing usage of the medication 614 by the patient 906.

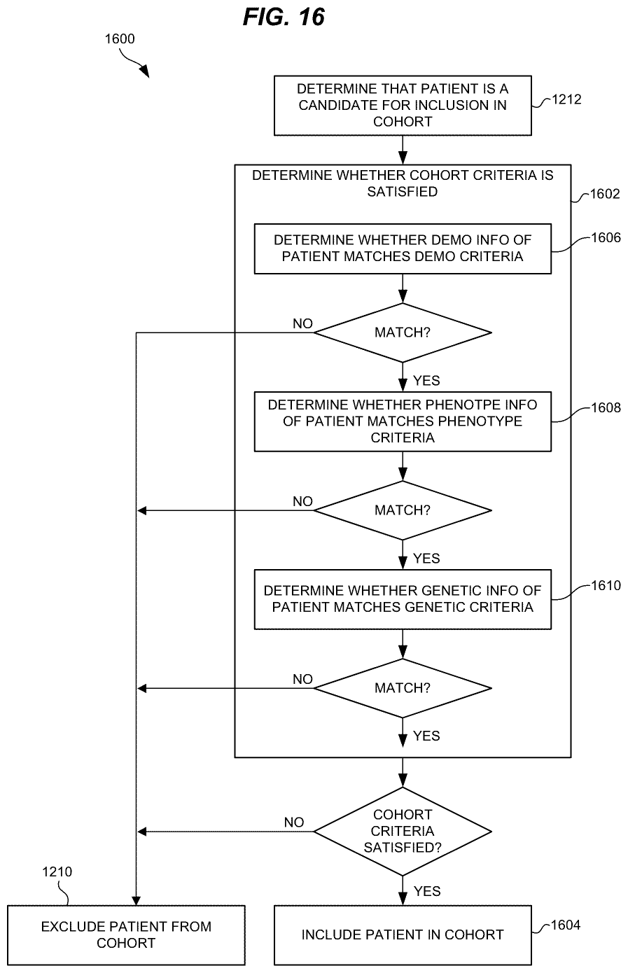

After the usage inference process 1200, medication insight controller 404 may further process the cohort criteria 616 to determine whether the patient 906 is eligible for inclusion in the cohort 904. FIG. 16 is a flow chart illustrating a method 1600 of processing cohort criteria 616 in an illustrative embodiment. It is noted that the order of the steps shown in FIG. 16 is provided as an example, and the steps may be performed in an alternative order. Before or after the usage inference process 1200, medication insight controller 404 determines whether cohort criteria 616 is satisfied for EHR data 116 of a patient 906 (step 1602). For example, medication insight controller 404 may determine whether demographic information of the patient 906 matches or satisfies demographic criteria 702 defined or specified in the cohort criteria 616 (step 1606). Medication insight controller 404 may then exclude the patient 906 from the cohort 904 when the demographic information of the patient 906 does not match the demographic criteria 702 (step 1210). Medication insight controller 404 may determine whether phenotype information of the patient 906 matches or satisfies phenotype criteria 704 defined or specified in the cohort criteria 616 (step 1608). Medication insight controller 404 may then exclude the patient 906 from the cohort 904 when phenotype information of the patient 906 does not match the phenotype criteria 704 (step 1210). Medication insight controller 404 may determine whether genetic information (e.g., sequencing data 142) of the patient 906 matches or satisfies genetic criteria 706 defined or specified in the cohort criteria 616 (step 1610). Medication insight controller 404 may then exclude the patient 906 from the cohort 904 when genetic information of the patient 906 does not match the genetic criteria 706 (step 1210). One technical benefit is the cohort 904 may be narrowed based on the cohort criteria 616 so that research may be performed on patients 906 having particular characteristics of interest.

When the cohort criteria 616 is satisfied, the patient 906 may be included in the cohort 904 (step 1604). Medication insight controller 404 performs similar examination of EHR data 116 for other patients 906 in the population 902 to determine which patients 906 are excluded from or included in the cohort 904. Medication insight controller 404 then compiles the EHR data 116 for the members of the cohort 904. In an embodiment, medication insight controller 404 may identify contact information for patients in the cohort 904 based on information in the EHRs 118, and trigger (automatically) a communication contacting the patients for participation in a clinical study or the like related to the medication 614.

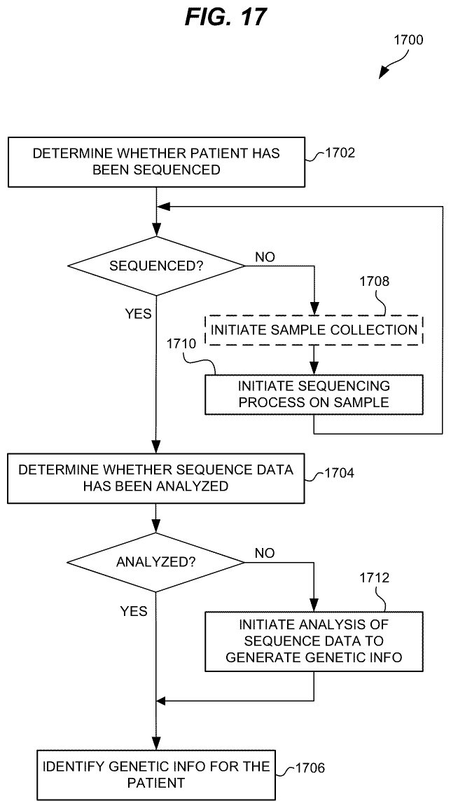

When processing the genetic criteria 706 for a patient 906, medication insight controller 404 may determine whether genetic information (e.g., sequencing data 142) exists for the patient 906. For example, the patient 906 may already have been sequenced, sequence data for the patient 906 may already be analyzed, etc., as part of a genomics service 132. FIG. 17 is a flow chart illustrating a method 1700 of genetic criteria processing in an illustrative embodiment. Medication insight controller 404 determines whether the patient 906 has been sequenced (step 1702). For example, medication insight controller 404 may determine whether (valid) raw sequence data 208 exists and/or is stored for the patient 906. When the patient 906 has been sequenced, medication insight controller 404 determines whether raw sequence data 208 for the patient 906 has been analyzed (step 1704). When the raw sequence data 208 for the patient 906 has been analyzed, sequencing data 142 (e.g., variant information) is stored for the patient 906, such as in data repository 406. Thus, medication insight controller 404 identifies the genetic information (i.e., sequencing data 142) from data repository 406 (step 1706), and is able to use the genetic information as a comparison with the genetic criteria 706 (see step 1610 in FIG. 16).

When the patient 906 has not been sequenced, medication insight controller 404 may take alternative steps to get the patient 906 sequenced. For example, patient 906 may not have provided a sample 204 to laboratory 136 for sequencing. Thus, medication insight controller 404 may interact with genomic data system 140 to initiate sample collection for the patient 906 (optional step 1708). To do so, genomic data system 140 may send a request to the patient 906, healthcare provider 102-104, etc., to provide a sample 204 of genetic material for the patient 906. If/when a sample 204 is received for the patient 906, genomic data system 140 may initiate a sequencing process on the sample 204 using sequencing equipment 134 (step 1710). Sequencing equipment 134 generates raw sequence data 208 for the patient 906, which may be stored in a data repository.

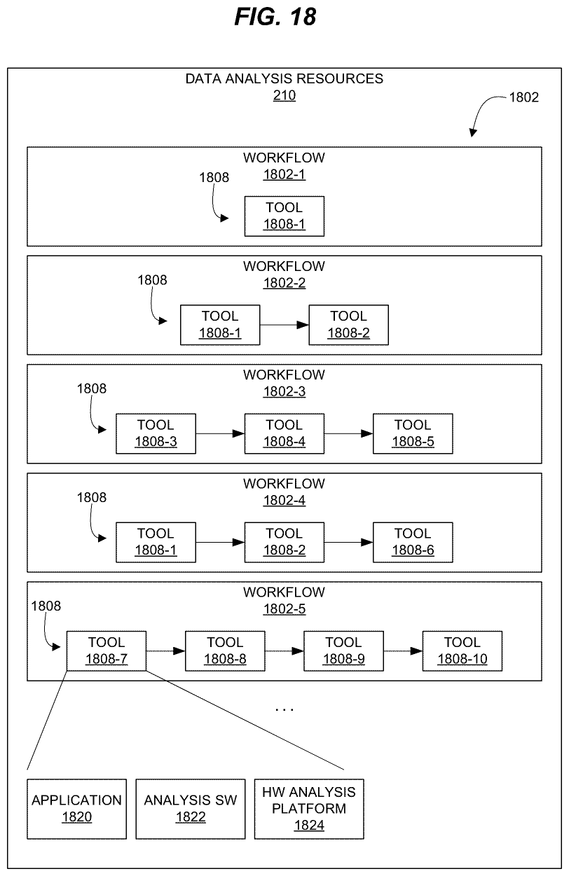

When the patient 906 has been sequenced but the raw sequence data 208 has not been analyzed, medication insight controller 404 may interact with genomic data system 140 to initiate analysis of the raw sequence data 208 to generate test results 212, referred to generally as genetic information for the patient 906 (step 1712). FIG. 18 is a block diagram illustrating data analysis resources 210 of genomic data system 140 in an illustrative embodiment. Data analysis resources 210 may support a plurality of workflows 1802 (e.g., workflows 1802-1, 1802-2, 1802-3, etc.), which may also be referred to as analysis pipelines, that are configured to perform genetic testing on raw sequence data 208. A workflow 1802 (or a portion of a workflow) comprises a set of one or more data processing elements that receives raw sequence data 208 as input, and outputs test results 212 for a genetic test. For example, a workflow 1802 may include one or more analysis tools 1808 (e.g., analysis tools 1808-1, 1808-2, 1808-3, etc.). An analysis tool 1808 is configured to extract insights from the raw sequence data 208. An analysis tool 1808 may comprise an application 1820, analysis software 1822, a hardware analysis platform 1824, etc. Each workflow 1802 may process the raw sequence data 208 differently to produce test results 212. When analyzing raw sequence data 208 for a patient 906, genomic data system 140 selects a workflow 1802 to analyze the raw sequence data 208 with one or more analysis tools 1808, such as alignment, variant calling, and/or any other analysis. A workflow 1802 may be selected based on a local policy or criteria, based on an order or request for a genetic test, etc. Genomic data system 140 then initiates the selected workflow 1802 to analyze the raw sequence data 208 for the patient 906 and generate test results 212. The test results 212, such as aligned sequence data, variant information or variant call data, etc., may be referred to generally as genetic information for the patient 906.

As described above regarding step 508 of FIG. 5A, medication insight controller 404 compiles the EHR data 116 for the cohort 904 of patients 906, and may further analyze the compiled EHR data 1112 for the cohort 904. In FIG. 5C, for example, medication insight controller 404 may perform electronic data processing on the compiled EHR data 1112 of the cohort 904 based on the analysis criteria 618 to generate analysis results 1114 (step 530). For example, the analysis criteria 618 may specify analysis of weight loss or changes in Body Mass Index (BMI) for patients 906 of the cohort 904, analysis of changes in blood sugar levels for patients 906 of the cohort 904, analysis of cardiovascular events or disorders for patients 906 of the cohort 904, analysis of symptoms or side-effects (e.g., chest pain, nausea, drowsiness, etc.) experienced by patients 906 of the cohort 904, etc. Thus, medication insight controller 404 may analyze the compiled EHR data 1112 based on the analysis criteria 618, and generate the desired analysis results 1114 provided to the requestor 606 in the report 1110 (optional step 532). One technical benefit is the coordination server 120 may provide a financially valuable data set to provide real-world evidence of drug safety and effectiveness, provide valuable phenotype/medication correlations that impact diagnosis and treatment processes, etc.

In one embodiment, medication insight controller 404 may use generated insights to provide recommendations for individual patients (optional step 534), based on patterns of behavior found in the analysis. These may include recommendations to a practitioner to provide medication fulfillment reminders. For example, if a substantial fraction (e.g., more than a quarter, more than half) of patients do not fill their first prescription for a specific medication (e.g., as indicated by an absence of a second prescription and/or a lack of a note in an EHR 118 indicating that the medication has been discontinued), medication insight controller 404 may provide a message via EHR system 114 that an additional reminder should be transmitted to a patient that has just received an order for their first prescription for that medication (e.g., as indicated within an EHR for that patient). In a further example, if medication insight controller 404 determines that a substantial fraction of patients have a gap between their first prescription order and second prescription order that is longer than the average prescription duration for the medication, then medication insight controller 404 may provide a message via EHR system 114 for a practitioner indicating that a patient with an active prescription order for the medication should be periodically reminded (e.g., once per week or once per month) to consistently use the medication.

In further embodiments, medication insight controller 404 provide guidelines and/or recommendations alongside discussion with a provider based on the specific situation for a patient. When relevant, patients might be flagged by medication insight controller 404 as potentially benefiting from a dose increase based on medication/lab data indicated in EHR system 114 for the patient. For example, for statins, there are different types of statins and different doses of each type, which can correspond to different intensity levels (low, moderate, or high intensity statin therapy). If a patient is not achieving LDL-cholesterol reduction goals on a moderate dose, then analytic work performed by medication insight controller 404 may flag the patient for being considered for an increased dose, via EHR system 114.

In instances for a specific medication in which patients may differentially be recommended a certain dose based on levels of laboratory or other measurements that are available in an EHR 118 (e.g., LDL-cholesterol concentration with respect to the dose of one's statin therapy), then analytic work may flag individuals who may possibly benefit from consideration of a dose increase, alongside discussion with one's provider, based on whether treatment goals have been met with respect to their current dose. Stated more generally, in an embodiment, medication insight controller 404 may provide one or more recommendations regarding usage of the medication 614 (optional step 534). For example, medication insight controller 404 may recommend a dosage of the medication 614, recommend a dosage change/adjustment of the medication, etc., for one or more of the patients in the cohort 904 to effect a particular treatment for a disease or medical condition. Medication insight controller 404 may implement a ML system 424 to generate or provide the recommendations. Medication insight controller 404 may provide the recommendation(s) to a healthcare provider 102-104, to individual patients, etc.

### Example

In the following example, additional processes, systems, and methods may be described in the context of medication research. The processes, systems, and methods described in this example may be incorporated in embodiments described above as desired.

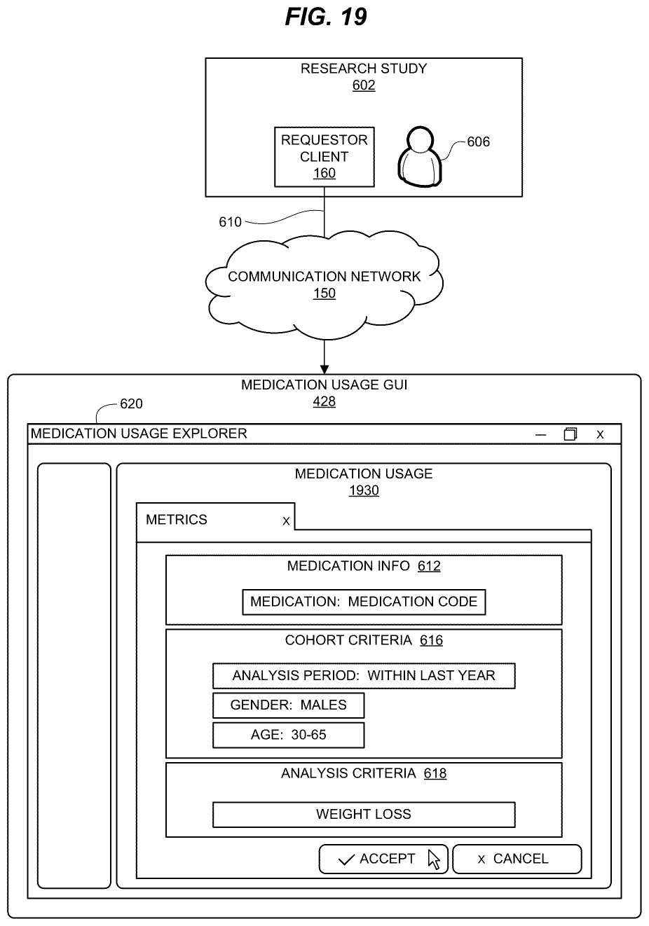

Medication insight controller 404 provides medication usage GUI 428 (i.e., medication usage explorer 620) that enables a requestor 606 to request research or analysis of a medication of interest. For example, the research study may be for a weight-loss drug, such as a semaglutide. FIG. 19 illustrates a medication usage GUI 428 in an illustrative embodiment. In this example, medication usage GUI 428 provides medication usage explorer 620 that allows the requestor 606 to interact with medication insight controller 404/medication usage explorer application 426 via communication network 150. Medication usage explorer 620 displays a medication usage graphical element 1930 that allows a requestor 606 to enter or input information of a medication of interest (i.e., medication code), and cohort criteria 616 to define a cohort 904 of patients 906. In this example, cohort criteria 616 may comprise an analysis period of “within the last year”, and demographic criteria 702 of “Males” within an age range of “30-65”, although other cohort criteria 616 may be defined, such as patients with no type 2 diabetes, minimum dosage information for the weight-loss drug, no indication of usage of certain other drugs, a baseline weight or BMI measurement along with at least one follow-up measurement, patients with or without certain genetic variants, etc. Medication usage graphical element 1930 also allows a requestor 606 to enter or input analysis criteria 618, such as an indication of weight loss or BMI change within the cohort 904, such as over one or more time periods (e.g., 0-3 months, 3-6 months, 6-9 months, 9-12 months, etc.).

Medication insight controller 404 gathers EHR data 116 from EHR systems 112-114 of one or more healthcare providers 102-104, for patients 906 that are prescribed the weight loss drug. Direct identifiers (e.g., patient names, social security numbers, etc.) may be removed from the EHR data 116 as submitted to coordination server 120. As described above, the EHR data 116 may be formatted in a standard OMOP CDM format, and sent over communication network 150 via secure file transfer protocol or otherwise encrypted. Medication insight controller 404 may transform the EHR data 116 from each healthcare provider 102-104 into a uniform OMOP version, and make a copy of the EHR data 116. Medication insight controller 404 may review the EHR data 116 to remove any direct identifiers (if included) and/or in-direct identifiers that remain in the EHR data 116.

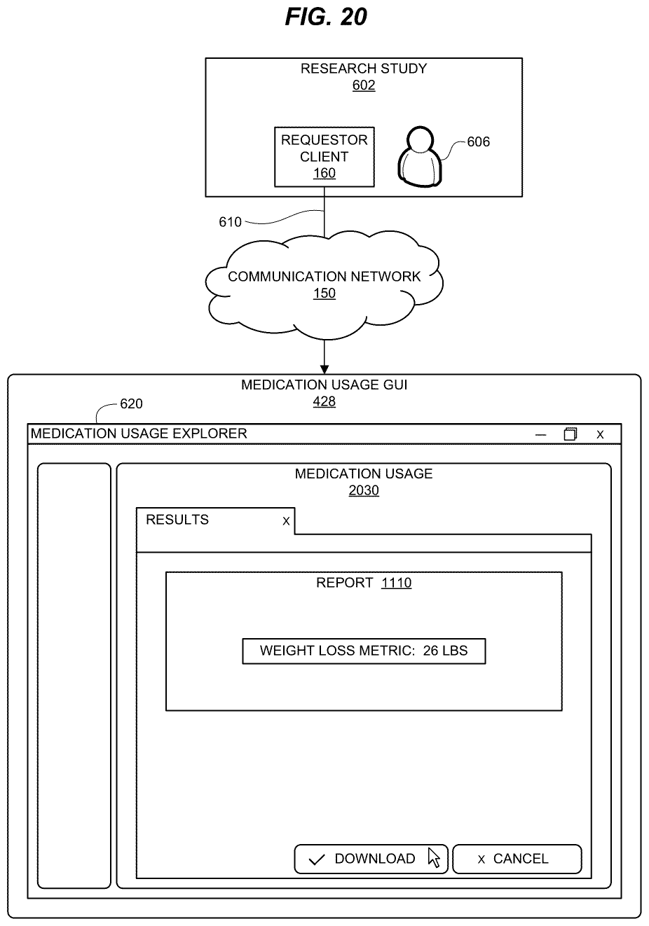

Medication insight controller 404 performs electronic data processing on the EHR data 116 for the patients 906 based on the cohort criteria 616 to identify a cohort 904 of patients 906. In other words, medication insight controller 404 processes the EHR data 116 to identify male patients between the ages of “30-65” that were prescribed the weight loss drug, which defines the cohort 904. Medication insight controller 404 compiles the EHR data 116 for the cohort 904 of patients 906, and then analyzes the compiled EHR data 1112 based on the analysis criteria 618 to generate a report 1110 of the analysis results 1114. Thus, medication insight controller 404 parses the EHR data 116 for the cohort 904 to determine a weight loss metric or BMI change metric (e.g., % reduction) for the patients 906 of the cohort 904. Medication insight controller 404 then provides the medication usage GUI 428 to display a report 1110 of the analysis results 1114. FIG. 20 illustrates a medication usage GUI 428 providing a report 1110 in an illustrative embodiment. In this example, medication usage GUI 428 again provides a medication usage explorer 620 that displays or provides a report 1110 regarding the cohort 1104. Medication usage explorer 620 displays a medication usage graphical element 2030 configured to display a weight loss metric, such as “26 lbs” indicating an average weight loss for the patients 906 of the cohort 904.

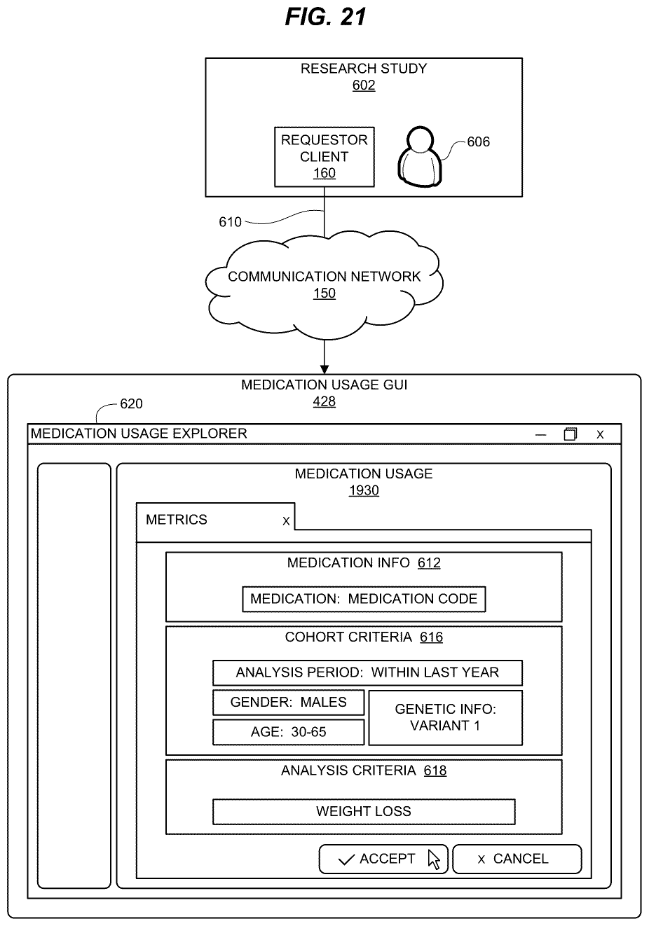

As described above, medication insight controller 404 may further consider genetic criteria 706 when defining the cohort 904. Medication insight controller 404 may therefore correlate sequencing data 142 of a patient 906 with the EHR data 116 of the patient 906. FIG. 21 illustrates a medication usage GUI 428 in an illustrative embodiment. In this example, the cohort criteria 616 further specifies genetic criteria 706 of “Variant 1”, which specifies that patients 906 of the cohort 904 also have a genetic variant generally referred to as “Variant 1”.

In general, laboratory procedures related to genetics may include accessioning, sample plating, storage, extraction, library preparation, enrichment, and sequencing processes. These processes acquire genetic material from a sample, separate the genetic material from other constituents, duplicate the genetic material, and quantify the genetic material order to determine a swathe of sequence data, such as an exome or entire genome for a subject (e.g., a human, an animal, a pathogen, an organelle, etc.).

Sequencing may be performed according to any of a variety of techniques, including short-read and long-read techniques. In one embodiment, the sequencing is performed as Sequencing by Synthesis (SBS) at genetic analyzer equipment. For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers. In one embodiment, the chemical identifiers comprise nucleotide sequences that are detectable during the sequencing process to determine a corresponding Laboratory Sample Identifier (LSI).

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

Sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence which can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome, or another set of genomic data, is being tested, different reagents (e.g., probes, primers, etc.) may be chosen. That is, different reagents may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

In some embodiments, genetic material is used for detection of a pathogen rather than for sequencing. Detecting a pathogen may involve the use of a real-time Polymerase Chain Reaction (PCR) system that performs PCR. The real-time PCR system may further add a reactive agent to individual wells of a library preparation microplate, that fluoresces when bound to genetic material for the pathogen. By analyzing fluorescence at known periods of time after PCR has initiated, presence of a pathogen is determined. Genetic testing for a pathogen may thereby forego sequencing in some embodiments.

Raw sequence data generated during synthesis may be stored in a file format, such as Binary Base Call (BCL), depending on the sequencing equipment used. This raw data may be fed to an analytical pipeline, such as a cloud-based computing environment. Raw sequence data may be processed by the analytical pipeline into a second format, such as a text-based FASTQ format, that reports the sequence information (i.e., the sequence reads) and corresponding quality scores. The second format is then analyzed to perform alignment of sequence reads to a reference genome, such as a reference genome reported in a Browser Extensible Data (BED) file. The aligned sequence data may be reported as a Binary Alignment Map (BAM) file. The aligned sequence data may then be called, resulting in a Variant Call Format (VCF) file reporting called variants at each location of the genome that was sequenced, together with secondary metrics, such as quality indicator metrics.

The called sequence data may be provided to a data analyst via a User Interface (UI), such as a GUI presented via a display. The technician may then validate the resulting called sequence data and release it for reporting to subjects, healthcare providers, and/or scientists.

Although specific embodiments were described herein, the scope of the invention is not limited to those specific embodiments. The scope of the invention is defined by the following claims and any equivalents thereof.

What is claimed is:

1. An apparatus, comprising:
a network interface configured to communicate over a communication network; and
a medication insight controller, comprising a processor and memory, configured to execute an algorithm to:
receive a request regarding a medication via the network interface;
obtain Electronic Health Record (EHR) data for a population of patients prescribed the medication within a target time period;
perform electronic data processing on the EHR data for the population of the patients based on cohort criteria specified in the request to identify a cohort of the patients from the population;
compile the EHR data for the cohort; and
provide a report regarding the medication based on the compiled EHR data via the network interface;
wherein to identify the cohort, the medication insight controller is configured to implement a usage inference process to estimate medication usage of a patient during the target time period based on prescription data included in the EHR data, the usage inference process comprising:
processing the prescription data to determine whether the prescription data indicates multiple prescriptions of the medication during the target time period, and excluding the patient from the cohort when the prescription data indicates less than the multiple prescriptions;
processing the prescription data to estimate a time gap between consecutive prescriptions, and excluding the patient from the cohort when the time gap exceeds a gap threshold; and
processing the prescription data to estimate a usage duration of the medication during the target time period, and excluding the patient from the cohort when the usage duration is less than a usage threshold.

2. The apparatus of claim 1, wherein the medication insight controller is further configured to execute the algorithm to:
perform electronic data processing on the compiled EHR data based on analysis criteria specified in the request to generate analysis results; and
provide the analysis results in the report.

3. The apparatus of claim 1, wherein the medication insight controller is further configured to execute the algorithm to:
provide a medication usage graphical user interface configured to receive the cohort criteria from a requestor to define the cohort from the population of the patients, and to provide the report based on the compiled EHR data.

4. The apparatus of claim 1, wherein:
the EHR data is formatted according to an Observational Medical Outcomes Partnership (OMOP) Common Data Model (CDM) standard.

5. The apparatus of claim 1, wherein:
the medication insight controller is configured to implement a machine-learning system to process the EHR data.

6. The apparatus of claim 1, wherein the medication insight controller, to estimate the time gap between the consecutive prescriptions, is configured to:
estimate a start date for a preceding prescription and a start date for a succeeding prescription; and
estimate the time gap between the start date for the preceding prescription and the start date for the succeeding prescription.

7. The apparatus of claim 1, wherein the medication insight controller, to estimate the time gap between the consecutive prescriptions, is configured to:
estimate an end date for a preceding prescription and a start date for a succeeding prescription; and
estimate the time gap between the end date for the preceding prescription and the start date for the succeeding prescription;
wherein to estimate the end date for the preceding prescription, the medication insight controller is configured to:
estimate a start date for the preceding prescription;
identify an expected prescription duration for the medication; and
estimate the end date of the preceding prescription as the start date plus the expected prescription duration.

8. The apparatus of claim 1, wherein the medication insight controller, to estimate the usage duration, is configured to:
estimate a start date for a first prescription within the target time period;
estimate an end date for a last prescription within the target time period; and
estimate the usage duration between the start date for the first prescription and the end date for the last prescription;
wherein to estimate the end date for the last prescription, the medication insight controller is configured to:
estimate a start date for the last prescription;
identify an expected prescription duration for the medication; and
estimate the end date of the last prescription as the start date for the last prescription plus the expected prescription duration.

9. The apparatus of claim 1, wherein the medication insight controller is further configured to:
determine whether demographic information of the patient matches demographic criteria specified in the cohort criteria; and
exclude the patient from the cohort when the demographic information does not match the demographic criteria.

10. The apparatus of claim 1, wherein the medication insight controller is further configured to:
determine whether phenotype information of the patient matches phenotype criteria specified in the cohort criteria; and
exclude the patient from the cohort when the phenotype information does not match the phenotype criteria.

11. The apparatus of claim 1, wherein the medication insight controller is further configured to:
determine whether genetic information of the patient matches genetic criteria specified in the cohort criteria; and
exclude the patient from the cohort when the genetic information does not match the genetic criteria.

12. A method, comprising:
executing an algorithm, at a medication insight controller comprising a processor and memory, to perform:
receiving a request regarding a medication over a communication network;
obtaining Electronic Health Record (EHR) data for a population of patients prescribed the medication within a target time period;
performing electronic data processing on the EHR data for the population of the patients based on cohort criteria specified in the request to identify a cohort of the patients from the population;
compiling the EHR data for the cohort; and
providing a report regarding the medication based on the compiled EHR data over the communication network;
wherein identifying the cohort comprises implementing a usage inference process to estimate medication usage of a patient during the target time period based on prescription data included in the EHR data, the usage inference process comprising:
processing the prescription data to determine whether the prescription data indicates multiple prescriptions of the medication during the target time period, and excluding the patient from the cohort when the prescription data indicates less than the multiple prescriptions;
processing the prescription data to estimate a time gap between consecutive prescriptions, and excluding the patient from the cohort when the time gap exceeds a gap threshold; and
processing the prescription data to estimate a usage duration of the medication during the target time period, and excluding the patient from the cohort when the usage duration is less than a usage threshold.

13. The method of claim 12, further comprising:
performing electronic data processing on the compiled EHR data based on analysis criteria specified in the request to generate analysis results; and
providing the analysis results in the report.

14. The method of claim 12, further comprising:
providing a medication usage graphical user interface configured to receive the cohort criteria from a requestor to define the cohort from the population of the patients, and to provide the report based on the compiled EHR data.

15. The method of claim 12, wherein the executing the algorithm comprises:
implementing a machine-learning system to process the EHR data.

16. The method of claim 12, wherein estimating the time gap between the consecutive prescriptions comprises:
estimating a start date for a preceding prescription and a start date for a succeeding prescription; and
estimating the time gap between the start date for the preceding prescription and the start date for the succeeding prescription.

17. The method of claim 12, wherein estimating the time gap between the consecutive prescriptions comprises:
estimating an end date for a preceding prescription and a start date for a succeeding prescription; and
estimating the time gap between the end date for the preceding prescription and the start date for the succeeding prescription;
wherein the estimating the end date for the preceding prescription comprises:
estimating a start date for the preceding prescription;
identifying an expected prescription duration for the medication; and
estimating the end date of the preceding prescription as the start date plus the expected prescription duration.

18. The method of claim 12, wherein the estimating the usage duration comprises:
estimating a start date for a first prescription within the target time period;
estimating an end date for a last prescription within the target time period; and
estimating the usage duration between the start date for the first prescription and the end date for the last prescription;
wherein the estimate the end date for the last prescription comprises:
estimating a start date for the last prescription;
identifying an expected prescription duration for the medication; and
estimating the end date of the last prescription as the start date for the last prescription plus the expected prescription duration.

19. The method of claim 12, further comprising:
determining whether demographic information of the patient matches demographic criteria specified in the cohort criteria; and
excluding the patient from the cohort when the demographic information does not match the demographic criteria.

20. The method of claim 12, further comprising:
determining whether phenotype information of the patient matches phenotype criteria specified in the cohort criteria; and
excluding the patient from the cohort when the phenotype information does not match the phenotype criteria.

21. The method of claim 12, further comprising:
determining whether genetic information of the patient matches genetic criteria specified in the cohort criteria; and
excluding the patient from the cohort when the genetic information does not match the genetic criteria.

22. A non-transitory computer readable medium embodying programmed instructions executed by a processor, wherein the instructions direct the processor to implement a method comprising:
executing an algorithm, at a medication insight controller comprising a processor and memory, to perform:
receiving a request regarding a medication over a communication network;
obtaining Electronic Health Record (EHR) data for a population of patients prescribed the medication within a target time period;
performing electronic data processing on the EHR data for the population of the patients based on cohort criteria specified in the request to identify a cohort of the patients from the population;
compiling the EHR data for the cohort; and
providing a report regarding the medication based on the compiled EHR data over the communication network;
wherein identifying the cohort comprises implementing a usage inference process to estimate medication usage of a patient during the target time period based on prescription data included in the EHR data, the usage inference process comprising:
processing the prescription data to determine whether the prescription data indicates multiple prescriptions of the medication during the target time period, and excluding the patient from the cohort when the prescription data indicates less than the multiple prescriptions;
processing the prescription data to estimate a time gap between consecutive prescriptions, and excluding the patient from the cohort when the time gap exceeds a gap threshold; and
processing the prescription data to estimate a usage duration of the medication during the target time period, and excluding the patient from the cohort when the usage duration is less than a usage threshold.
